SEQUENCE LISTING (7 sequences)

SEQ ID NO: 1 - DNA, 53 bp, ARTIFICIAL
  Spacer region
taatacgactcactataggaaaaaaaaaaaaaaaaaaaaaggaggtatacatg

SEQ ID NO: 2 - protein, 8 aa, Artificial
  FLAG tag
DYKDDDDK

SEQ ID NO: 3 - protein, 6 aa, Artificial
  HIS6 tag
HHHHHH

SEQ ID NO: 4 - DNA, 15 bp, ARTIFICIAL
  PCR primer
aaggaggtatacatg

SEQ ID NO: 5 - DNA, 18 bp, ARTIFICIAL
  PCR primer
gtcgtcgtccttgtagtc

SEQ ID NO: 6 - DNA, 53 bp, ARTIFICIAL; identical to SEQ ID NO: 1
  PCR primer

SEQ ID NO: 7 - DNA, 57 bp, Artificial
  PCR primer
ttaaatagcggatgcgtggtggtggtgatggtgcttgtcgtcgtcgtccttgtagtc

(19) United States
(12) Patent Application Publication
(10) Pub. No.: US 2014/0256589 A1
(43) Pub. Date: Sep. 11, 2014

(54) METHODS FOR ACCELERATED SELECTION OF POLYPEPTIDES

(71) Applicant: Bristol-Myers Squibb Company, Princeton, NJ (US)

(72) Inventor: David A. Fabrizio, South Hamilton, MA (US)

(21) Appl. No.: 14/204,511

(22) Filed: Mar. 11, 2014

Related U.S. Application Data
(60) Provisional application No. 61/776,147, filed on Mar. 11, 2013.

(51) Int. Cl. C12P 21/00

(52) U.S. Cl. CPC C12P 21/005
USPC 506/9; 506/26

(57) ABSTRACT

In certain embodiments, the disclosure provides a method for generating an mRNA-protein fusion molecule. In other embodiments, the disclosure provides a method for selecting a desired polypeptide.

### CROSS-REFERENCE TO RELATED APPLICATIONS

This application claims the benefit of U.S. Provisional Application No. 61/776,147 filed Mar. 11, 2013, whose contents are hereby incorporated by reference.

### BACKGROUND OF THE INVENTION

In vitro selection and directed evolution of proteins can be achieved by linking phenotype (polypeptides) and genotype (nucleic acids). For example, in vitro cell-free construction of larger libraries by linking mRNA with its nascent polypeptide has been achieved by at least two display systems: (a) ribosome display systems (e.g., Hanes and Pliickthun, 1997, Proc. Natl. Acad. Sci. USA, 94, 4937-4942 5; He and Taussig, 1997, Nucleic Acids Res., 25, 5132-5134); (b) and mRNA display systems (e.g., Nemoto et al., 1997, FEBS Lett., 414, 405-408 8; Roberts and Szostak, 1997, Proc. Natl. Acad. Sci. USA, 94, 12297-12302). In addition, mRNA display systems have been improved to increase stability by fusing polypeptides with their encoding cDNAs such that polypeptides may be screened and selected while conjugated with their encoding cDNAs (e.g., Kurz et al., 2001, Chembiochem, 2, 666-672; Tabuchi et al., 2001, FEBS Lett., 508, 309-312). mRNA display systems have been used to select polypeptides (such as antibodies) with the highest affinity, specificity, stability, and/or other desirable characteristics.

Current in vitro mRNA display systems are time-consuming and require many different process steps. Therefore, there is a need to develop a rapid, simplified, and more efficient mRNA display method for accelerated selection of polypeptides.

### SUMMARY OF THE INVENTION

In certain embodiments, the present invention provides a method for generating an mRNA-protein fusion molecule, comprising: (a) contacting an in vitro transcription translation (IVTT) system with (1) a DNA molecule which comprises a protein coding sequence and a 5′ untranslated region (5′ UTR); and (2) a linker molecule which comprises (i) a nucleic acid portion which hybridizes to the 3′ end of the mRNA encoded by the DNA molecule; (ii) a peptide acceptor; and (iii) a psoralen moiety; (b) transcribing the DNA molecule into an mRNA and translating the mRNA into a protein in the IVTT system, wherein the protein is fused to the mRNA through the linker molecule; and (c) cross-linking the mRNA-protein fusion through the psoralen moiety. Optionally, the method further comprises reverse-transcribing the mRNA fused to the protein, thereby generating the DNA-mRNA-protein fusion molecule. For example, the IVTT system is an Escherichia coli based IVTT system. For example, the peptide acceptor is puromycin. In a specific example, the psoralen moiety is positioned at the 5′ terminus of the linker molecule and the peptide acceptor is positioned at the 3′ terminus of the linker molecule. Optionally, the linker molecule further comprises a non-nucleotide moiety (e.g., a C6 alkyl chain). Optionally, the cross-linking is achieved by UV light irradiation. In certain specific aspects, the protein is an antibody or an antibody fragment. In certain specific aspects, the transcription, translation, and cross-linking steps are performed in the same container. Optionally, certain purification steps are omitted. For example, the mRNA produced by transcription is not purified before translation step. For example, the mRNA-protein fusion is not purified before the reverse transcription step. Optionally, all the steps (e.g., transcription, translation, cross-linking, and reverse transcription) are carried out in one pot, and optionally take no more than 4 hours (for example, about 2, 3 or 4 hours), and optionally no more than 3 hours. In certain specific aspects, the DNA molecule further comprises at least a nucleic acid sequence which encodes at least one tag (e.g., histidine and/or FLAG) which facilities subsequent purification (e.g., after reverse transcription).

In certain embodiments, the present invention provides a method for generating an mRNA-protein fusion molecule, comprising: (a) contacting an in vitro transcription translation (IVTT) system which is deficient in one or more translation components with (1) a DNA molecule which comprises a protein coding sequence and a 5′ untranslated region (5′ UTR); and (2) a linker molecule which comprises (i) a nucleic acid portion which hybridizes to the 3′ end of the mRNA encoded by the DNA molecule; (ii) a peptide acceptor; and (iii) a psoralen moiety; (b) transcribing the DNA molecule into an mRNA, wherein the linker molecule anneals to the 3′ end of the mRNA; (c) cross-linking the linker molecule and the mRNA through the psoralen moiety; and (d) translating the mRNA into a protein by adding the one or more translation components into the IVTT system, wherein the protein is fused to the mRNA through the linker molecule. Optionally, the method further comprises reverse-transcribing the mRNA fused to the protein, thereby generating the DNA-mRNA-protein fusion molecule. For example, the IVTT system is an Escherichia coli based IVTT system. To illustrate, the IVTT system is deficient in one or more translation components selected from amino acids, tRNAs, and ribosomes. For example, the peptide acceptor is puromycin. In a specific example, the psoralen moiety is positioned at the 5′ terminus of the linker molecule and the peptide acceptor is positioned at the 3′ terminus of the linker molecule. Optionally, the linker molecule further comprises a non-nucleotide moiety (e.g., a C6 alkyl chain). Optionally, the cross-linking is achieved by UV light irradiation. In certain specific aspects, the protein is an antibody or an antibody fragment. In certain specific aspects, the transcription, translation, and cross-linking steps are performed in the same container. Optionally, certain purification steps are omitted. For example, the mRNA produced by transcription is not purified before translation. For example, the mRNA-protein fusion is not purified before the reverse transcription step. Optionally, all the steps (e.g., transcription, translation, cross-linking, and reverse transcription) are carried out in one pot, and optionally take no more than 4 hours (for example, about 2, 3 or 4 hours), and optionally no more than 3 hours. In certain specific aspects, the DNA molecule further comprises at least a nucleic acid sequence which encodes at least one tag (e.g., histidine and/or FLAG) which facilities subsequent purification (e.g., after reverse transcription).

In certain embodiments, the present invention provides a method for selecting a desired protein, comprising: (a) contacting an in vitro transcription translation (IVTT) system with: (1) a population of DNA molecules, each of which comprises a protein coding sequence and a 5′ untranslated region (5′ UTR); and (2) a linker molecule which comprises (i) a nucleic acid portion which hybridizes to the 3′ end of each of the mRNAs encoded by the DNA molecules; (ii) a peptide acceptor; and (iii) a psoralen moiety; (b) transcribing the population of DNA molecules into a population of mRNAs and translating the population of mRNAs into a population of proteins in the IVTT system, wherein each protein is fused to its encoding mRNA; (c) cross-linking the mRNA-protein fusions through the psoralen moiety; and (d) reverse-transcribing the mRNAs, thereby generating the DNA-mRNA-protein fusions; and (e) selecting a desired mRNA-protein fusion, thereby selecting the desired protein. For example, the IVTT system is an Escherichia coli based IVTT system. For example, the peptide acceptor is puromycin. In a specific example, the psoralen moiety is positioned at the 5′ terminus of the linker molecule and the peptide acceptor is positioned at the 3′ terminus of the linker molecule. Optionally, the linker molecule further comprises a non-nucleotide moiety (e.g., a C6 alkyl chain). Optionally, the cross-linking is achieved by UV light irradiation. In certain specific aspects, the protein is an antibody or an antibody fragment. In certain specific aspects, the transcription, translation, and cross-linking steps are performed in the same container. Optionally, certain purification steps are omitted. For example, the mRNAs produced by transcription are not purified before translation. For example, the mRNA-protein fusions are not purified before the reverse transcription step. In certain specific embodiments, the transcription, translation, cross-linking, and reverse transcription steps are carried out in one pot, and optionally take no more than 4 hours (for example, about 2, 3 or 4 hours), and optionally no more than 3 hours. In certain specific aspects, each of the DNA molecules further comprises at least a nucleic acid sequence which encodes at least one tag (e.g., histidine and/or FLAG) which facilities subsequent purification (e.g., after reverse transcription). Optionally, the mRNA-protein fusions are purified after the reverse transcription step and before the selection step.

In certain embodiments, the present invention provides a method for selecting a desired protein, comprising: (a) contacting an in vitro transcription translation (IVTT) system which is deficient in one or more translation components, with: (1) a population of DNA molecules, each of which comprises a protein coding sequence and a 5′ untranslated region (5′ UTR); and (2) a linker molecule which comprises (i) a nucleic acid portion which hybridizes to the 3′ end of each of the mRNAs encoded by the DNA molecules; (ii) a peptide acceptor; and (iii) a psoralen moiety; (b) transcribing the population of DNA molecules into a population of mRNAs in the IVTT system, wherein the linker molecule anneals to the 3′ end of each of mRNAs; (c) cross-linking the linker molecule and each of the mRNAs through the psoralen moiety; and (d) translating the population of mRNAs into a population of proteins by adding the one or more translation components into the IVTT system, wherein each protein is fused to its encoding mRNA; and (e) reverse-transcribing the mRNAs, thereby generating the DNA-mRNA-protein fusions; and (f) selecting a desired mRNA-protein fusion, thereby selecting the desired protein. For example, the IVTT system is an Escherichia coli based IVTT system. To illustrate, the IVTT system is deficient in one or more translation components selected from amino acids, tRNAs, and ribosomes. For example, the peptide acceptor is puromycin. In a specific example, the psoralen moiety is positioned at the 5′ terminus of the linker molecule and the peptide acceptor is positioned at the 3′ terminus of the linker molecule. Optionally, the linker molecule further comprises a non-nucleotide moiety (e.g., a C6 alkyl chain). Optionally, the cross-linking is achieved by UV light irradiation. In certain specific aspects, the protein is an antibody or an antibody fragment. In certain specific aspects, the transcription, translation, and cross-linking steps are performed in the same container. Optionally, certain purification steps are omitted. For example, the mRNAs produced by transcription are not purified before translation. For example, the mRNA-protein fusions are not purified before the reverse transcription step. In certain specific embodiments, the transcription, translation, cross-linking, and reverse transcription steps are carried out in one pot, and optionally take no more than 4 hours (for example, about 2, 3 or 4 hours), and optionally no more than 3 hours. In certain specific aspects, each of the DNA molecules further comprises at least a nucleic acid sequence which encodes at least one tag (e.g., histidine and/or FLAG) which facilities subsequent purification (e.g., after reverse transcription). Optionally, the mRNA-protein fusions are purified after the reverse transcription step and before the selection step.

### BRIEF DESCRIPTION OF THE DRAWINGS

FIG. 1A shows an exemplary mRNA display selection cycle. A library of double-stranded DNA sequences is transcribed to generate mRNA (a). The mRNA is purified (b), then ligated to a puromycin oligonucleotide (c), and used to program an in vitro translation reaction (d). The resulting mRNA-protein fusion is purified (e), and cDNA synthesis is performed through reverse transcription (f). A final protein tag based purification is performed (g), and the cDNA/mRNA-protein fusion is then selected using the target of interest (h). PCR is used to regenerate the full-length DNA construct (i).

FIG. 1B shows the accelerated selection cycle. A library of double-stranded DNA sequences is transcribed to generate mRNA, subsequently ligated to a puromycin oligonucleotide, and translated into its nascent polypeptide (a). The non-purified material is subsequently reverse-transcribed to generate cDNA (a). A protein tag based purification is performed (b). The cDNA/mRNA-protein fusion is then selected using the target of interest (c). PCR is used to regenerate the full-length DNA construct (d).

FIG. 2 shows the key differences between the ASCENT protocol and the standard PROfusion™ protocol.

FIG. 3 shows the construction of an Adnectin library by extension of overlapping oligonucleotides.

FIG. 4 shows a standard curve for the qPCR analysis.

FIG. 5A shows the comparison of the mRNA-protein fusion yields from the ASCENT protocol and the standard PROfusion™ protocol.

FIG. 5B shows that ASCENT library size scaled with the IVTT volume.

FIG. 6 shows the results on enrichment rate of adnectin binders using the ASCENT-XLC protocol.

### DETAILED DESCRIPTION OF THE INVENTION

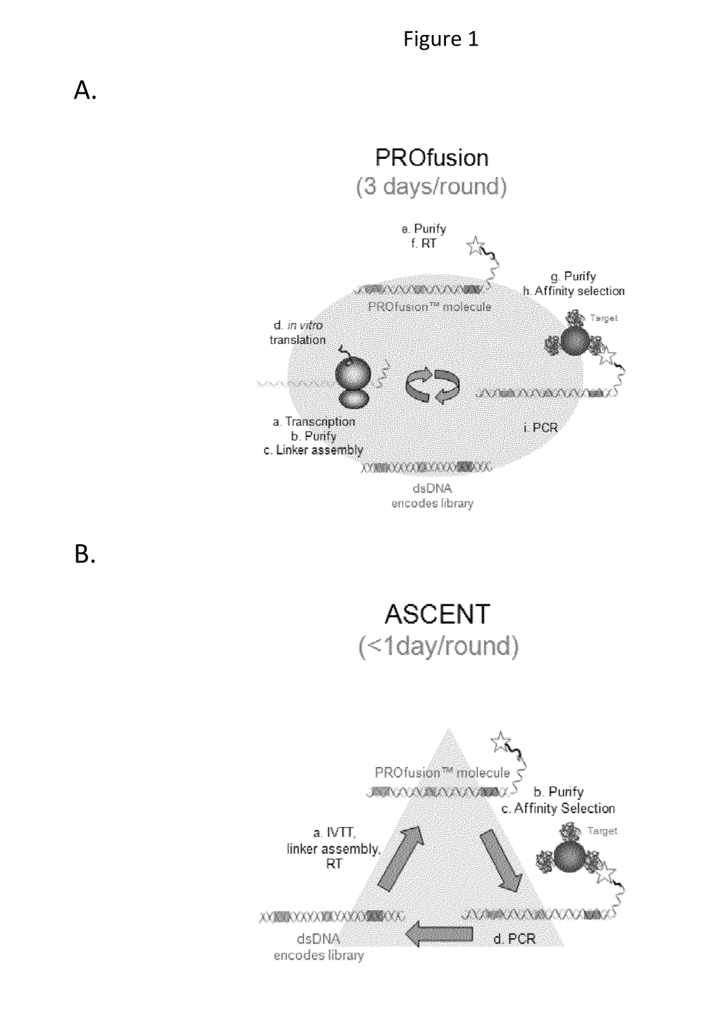

In certain aspects, the present invention is directed to a method for rapidly generating an mRNA-protein fusion molecule, and a method for rapidly selecting a desired protein. In a specific embodiment, the present invention features an improved mRNA display method referred to as “ASCENT” (Accelerated Selection of Engineered Therapeutics). An exemplary mRNA display experiment (e.g., PROfusion™) is shown in FIG. 1. Briefly, a DNA molecule is transcribed to produce an mRNA, and then a synthetic oligonucleotide containing a 3′ puromycin is ligated to the 3′ end of an mRNA. The mRNA product is then translated in rabbit reticulocyte lysate to produce a polypeptide which is covalently attached to its encoding mRNA. The mRNA-protein fusion is purified and proceeds to reverse transcription to form a cDNA/mRNA-protein fusion. The cDNA/mRNA-protein fusion is then selected using a target of interest. The selected cDNA/mRNA-protein fusion can then be PCR-amplified, and optionally subjected to additional rounds of selection. Detailed descriptions of experiments and protocols of mRNA display methods (e.g., PROfusion™) are previously described (see, e.g., Kurz et al., 2001, Chembiochem, 2, 666-672; Tabuchi et al., 2001, FEBS Lett., 508, 309-312; Barrick et al., 2001, Methods 23, 287-293; Kurz and Lohse, 2000, Molecules 5, 1259-1264; Kurz et al., 2000, Nucleic Acids Res. 28, E83; and Liu et al., 2000, Methods Enzymol. 318, 268-293).

As described in the working examples, ASCENT has various advantages over the standard mRNA display method such as PROfusion™. The ASCENT method utilizes a cell-free IVTT mixture which is reconstituted from the purified components necessary for E. coli transcription and translation components (e.g., PURExpress technology from New England Biolabs) in order to combine in vitro transcription and translation (IVTT) into a single step reaction, requiring only a single purification step before the selection (after reverse transcription). In addition, a modified version of ASCENT (termed ASCENT-XLC) utilizes a cell-free IVTT mixture, wherein certain translation components (e.g., tRNA and amino acid mix) are omitted from the original mixture but can be added into the mixture later. The ASCENT-XLC protocol affords greater control of the IVTT and photo-crosslinking reactions. Due to the pure nature of the IVTT reaction, the reverse transcription (RT) reaction may be carried out immediately thereafter, without the need for a purification step after the IVTT reaction, which is required in the standard PROfusion™ protocol in the form of an oligo-dT based purification. After the RT reaction, the library of mRNA-protein fusion molecules requires just a single purification step in the form of a Ni-NTA agarose based purification, which requires a HIS6 tag on the protein, or an anti-FLAG antibody based purification, which requires the FLAG tag sequence on the protein. The purified library can then be selected against a target of interest, and surviving molecules are amplified by PCR and subjected to further rounds of selection. This accelerated method of mRNA display (termed ASCENT) allows a single round of selection to be easily completed within a day, and entire selections in no more than a week, without any significant loss of yield and at a fraction of the cost of standard mRNA display such as PROfusion™. Moreover, because all of the reactions are done in a single tube, the potential for high throughput selections with the use of automation is very high.

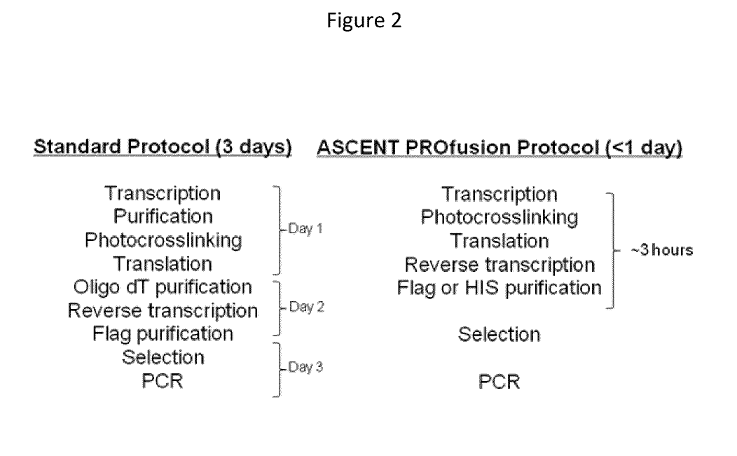

FIG. 2 shows the key differences between the ASCENT protocol and the standard PROfusion™ protocol. Specifically, ASCENT significantly reduces the selection time. For example, ASCENT combines the in vitro transcription and translation into a single step, and removes the two purification steps (one after transcription and the other after translation). As such, it takes only about three hours from transcription to post-RT purification (e.g., FLAG or HIS purification) according to the ASCENT method. By contrast, it takes about two days from transcription to post-RT purification (e.g., FLAG or HIS purification) according to the PROfusion™ method. In addition, the ASCENT method has the advantages of lower cost and high potential for automation, compared to the PROfusion™ method.

In certain embodiments, the present invention provides a method for generating an mRNA-protein fusion molecule, comprising: (a) contacting an in vitro transcription translation (IVTT) system with (1) a DNA molecule which comprises a protein coding sequence and a 5′ untranslated region (5′ UTR); and (2) a linker molecule which comprises (i) a nucleic acid portion which hybridizes to the 3′ end of the mRNA encoded by the DNA molecule; (ii) a peptide acceptor; and (iii) a psoralen moiety; (b) transcribing the DNA molecule into an mRNA and translating the mRNA into a protein in the IVTT system, wherein the protein is fused to the mRNA through the linker molecule; and (c) cross-linking the mRNA-protein fusion through the psoralen moiety. Optionally, the method further comprises reverse-transcribing the mRNA fused to the protein, thereby generating the DNA-mRNA-protein fusion molecule. For example, the IVTT system is an Escherichia coli based IVTT system. For example, the peptide acceptor is puromycin. In a specific example, the psoralen moiety is positioned at the 5′ terminus of the linker molecule and the peptide acceptor is positioned at the 3′ terminus of the linker molecule. Optionally, the linker molecule further comprises a non-nucleotide moiety (e.g., a C6 alkyl chain). Optionally, the cross-linking is achieved by UV light irradiation. In certain specific aspects, the protein is an antibody or an antibody fragment. In certain specific aspects, the transcription, translation, and cross-linking steps are performed in the same container. Optionally, certain purification steps are omitted. For example, the mRNA produced by transcription is not purified before translation step. For example, the mRNA-protein fusion is not purified before the reverse transcription step. In certain specific embodiments, all the steps (e.g., transcription, translation, cross-linking, and reverse transcription) are carried out in one pot, and optionally take no more than 4 hours (for example, about 2, 3, or 4 hours), and optionally no more than 3 hours. In certain specific aspects, the DNA molecule further comprises at least a nucleic acid sequence which encodes at least one tag (e.g., histidine and/or FLAG) which facilities subsequent purification (e.g., after reverse transcription).

In certain embodiments, the present invention provides a method for generating an mRNA-protein fusion molecule, comprising: (a) contacting an in vitro transcription translation (IVTT) system which is deficient in one or more translation components with (1) a DNA molecule which comprises a protein coding sequence and a 5′ untranslated region (5′ UTR); and (2) a linker molecule which comprises (i) a nucleic acid portion which hybridizes to the 3′ end of the mRNA encoded by the DNA molecule; (ii) a peptide acceptor; and (iii) a psoralen moiety; (b) transcribing the DNA molecule into an mRNA, wherein the linker molecule anneals to the 3′ end of the mRNA; (c) cross-linking the linker molecule and the mRNA through the psoralen moiety; and (d) translating the mRNA into a protein by adding the one or more translation components into the IVTT system, wherein the protein is fused to the mRNA through the linker molecule. Optionally, the method further comprises reverse-transcribing the mRNA fused to the protein, thereby generating the DNA-mRNA-protein fusion molecule. For example, the IVTT system is an Escherichia coli based IVTT system. To illustrate, the IVTT system is deficient in one or more translation components selected from amino acids, tRNAs, and ribosomes. For example, the peptide acceptor is puromycin. In a specific example, the psoralen moiety is positioned at the 5′ terminus of the linker molecule and the peptide acceptor is positioned at the 3′ terminus of the linker molecule. Optionally, the linker molecule further comprises a non-nucleotide moiety (e.g., a C6 alkyl chain). Optionally, the cross-linking is achieved by UV light irradiation. In certain specific aspects, the protein is an antibody or an antibody fragment. In certain specific aspects, the transcription, translation, and cross-linking steps are performed in the same container. Optionally, certain purification steps are omitted. For example, the mRNA produced by transcription is not purified before translation. For example, the mRNA-protein fusion is not purified before the reverse transcription step. In certain specific embodiments, all the steps (e.g., transcription, translation, cross-linking, and reverse transcription) are carried out in one pot, and optionally take no more than 4 hours (for example, about 2, 3, or 4 hours), and optionally no more than 3 hours. In certain specific aspects, the DNA molecule further comprises at least a nucleic acid sequence which encodes at least one tag (e.g., histidine and/or FLAG) which facilities subsequent purification (e.g., after reverse transcription).

In certain embodiments, the present invention provides a method for selecting a desired protein, comprising: (a) contacting an in vitro transcription translation (IVTT) system with: (1) a population of DNA molecules, each of which comprises a protein coding sequence and a 5′ untranslated region (5′ UTR); and (2) a linker molecule which comprises (i) a nucleic acid portion which hybridizes to the 3′ end of each of the mRNAs encoded by the DNA molecules; (ii) a peptide acceptor; and (iii) a psoralen moiety; (b) transcribing the population of DNA molecules into a population of mRNAs and translating the population of mRNAs into a population of proteins in the IVTT system, wherein each protein is fused to its encoding mRNA; (c) cross-linking the mRNA-protein fusions through the psoralen moiety; and (d) reverse-transcribing the mRNAs, thereby generating the DNA-mRNA-protein fusions; and (e) selecting a desired mRNA-protein fusion, thereby selecting the desired protein. For example, the IVTT system is an Escherichia coli based IVTT system. For example, the peptide acceptor is puromycin. In a specific example, the psoralen moiety is positioned at the 5′ terminus of the linker molecule and the peptide acceptor is positioned at the 3′ terminus of the linker molecule. Optionally, the linker molecule further comprises a non-nucleotide moiety (e.g., a C6 alkyl chain). Optionally, the cross-linking is achieved by UV light irradiation. In certain specific aspects, the protein is an antibody or an antibody fragment. In certain specific aspects, the transcription, translation, and cross-linking steps are performed in the same container. Optionally, certain purification steps are omitted. For example, the mRNAs produced by transcription are not purified before translation. For example, the mRNA-protein fusions are not purified before the reverse transcription step. In certain specific embodiments, the transcription, translation, cross-linking, and reverse transcription steps are carried out in one pot, and optionally take no more than 4 hours (for example, about 2, 3, or 4 hours), and optionally no more than 3 hours. In certain specific aspects, each of the DNA molecules further comprises at least a nucleic acid sequence which encodes at least one tag (e.g., histidine and/or FLAG) which facilities subsequent purification (e.g., after reverse transcription). Optionally, the mRNA-protein fusions are purified after the reverse transcription step and before the selection step.

In certain embodiments, the present invention provides a method for selecting a desired protein, comprising: (a) contacting an in vitro transcription translation (IVTT) system which is deficient in one or more translation components, with: (1) a population of DNA molecules, each of which comprises a protein coding sequence and a 5′ untranslated region (5′ UTR); and (2) a linker molecule which comprises (i) a nucleic acid portion which hybridizes to the 3′ end of each of the mRNAs encoded by the DNA molecules; (ii) a peptide acceptor; and (iii) a psoralen moiety; (b) transcribing the population of DNA molecules into a population of mRNAs in the IVTT system, wherein the linker molecule anneals to the 3′ end of each of mRNAs; (c) cross-linking the linker molecule and each of the mRNAs through the psoralen moiety; and (d) translating the population of mRNAs into a population of proteins by adding the one or more translation components into the IVTT system, wherein each protein is fused to its encoding mRNA; and (e) reverse-transcribing the mRNAs, thereby generating the DNA-mRNA-protein fusions; and (f) selecting a desired mRNA-protein fusion, thereby selecting the desired protein. For example, the IVTT system is an Escherichia coli based IVTT system. To illustrate, the IVTT system is deficient in one or more translation components selected from amino acids, tRNAs, and ribosomes. For example, the peptide acceptor is puromycin. In a specific example, the psoralen moiety is positioned at the 5′ terminus of the linker molecule and the peptide acceptor is positioned at the 3′ terminus of the linker molecule. Optionally, the linker molecule further comprises a non-nucleotide moiety (e.g., a C6 alkyl chain). Optionally, the cross-linking is achieved by UV light irradiation. In certain specific aspects, the protein is an antibody or an antibody fragment. In certain specific aspects, the transcription, translation, and cross-linking steps are performed in the same container. Optionally, certain purification steps are omitted. For example, the mRNAs produced by transcription are not purified before translation. For example, the mRNA-protein fusions are not purified before the reverse transcription step. In certain specific embodiments, the transcription, translation, cross-linking, and reverse transcription steps are carried out in one pot, and optionally take no more than 4 hours (for example, about 2, 3, or 4 hours), and optionally no more than 3 hours. In certain specific aspects, each of the DNA molecules further comprises at least a nucleic acid sequence which encodes at least one tag (e.g., histidine and/or FLAG) which facilities subsequent purification (e.g., after reverse transcription). Optionally, the mRNA-protein fusions are purified after the reverse transcription step and before the selection step.

By a “protein” is meant any two or more naturally occurring or modified amino acids joined by one or more peptide bonds. “Protein”, “polypeptide”, and “peptide” are used interchangeably herein.

By a “RNA” is meant a sequence of two or more covalently bonded, naturally occurring or modified ribonucleotides. One example of a modified RNA included within this term is phosphorothioate RNA. “RNA” and “mRNA” are used interchangeably herein.

As used herein, by a “population” of molecules is meant more than one molecule (for example, more than one RNA, DNA, or RNA-protein fusion molecule). Because the methods of the invention facilitate selections which begin, if desired, with large numbers of candidate molecules, a “population” of molecules according to the invention preferably means more than 10⁹ molecules, more preferably, more than 10¹⁰, 10¹¹, or 10¹² molecules, and, most preferably, more than 10¹³ molecules.

By “selecting” is meant substantially partitioning a molecule from other molecules in a population. As used herein, a “selecting” step provides at least a 2-fold, preferably, a 30-fold, more preferably, a 100-fold, and, most preferably, a 1000-fold enrichment of a desired molecule relative to undesired molecules in a population following the selection step. As indicated herein, a selection step may be repeated any number of times, and different types of selection steps may be combined in a given approach.

### Peptide Acceptors

By a “peptide acceptor” is meant any molecule capable of being added to the C-terminus of a growing protein chain by the catalytic activity of the ribosomal peptidyl transferase function. Typically, such molecules contain (i) a nucleotide or nucleotide-like moiety, for example, adenosine or an adenosine analog (di-methylation at the N-6 amino position is acceptable); (ii) an amino acid or amino acid-like moiety, such as any of the D- or L-amino acids or any amino acid analog thereof including O-methyl tyrosine or any of the analogs described by Ellman et al. (Meth. Enzymol. 202:301, 1991); and (iii) a linkage between the two (for example, an ester, amide, or ketone linkage at the 3′ position or, less preferably, the 2′ position). Preferably, this linkage does not significantly perturb the pucker of the ring from the natural ribonucleotide conformation. Peptide acceptors may also possess a nucleophile, which may be, without limitation, an amino group, a hydroxyl group, or a sulfhydryl group. In addition, peptide acceptors may be composed of nucleotide mimetics, amino acid mimetics, or mimetics of the combined nucleotide-amino acid structure.

Although an exemplary peptide acceptor is puromycin, other compounds that act in a manner similar to puromycin may be used. Other possible choices for protein acceptors include pyrazolopyrimidine or any related derivatives and tRNA-like structures, and other compounds known in the art. Such compounds include, without limitation, any compound which possesses an amino acid linked to an adenine or an adenine-like compound, such as the amino acid nucleotides, phenylalanyl-adenosine (A-Phe), tyrosyl adenosine (A-Tyr), and alanyl adenosine (A-Ala), as well as amide-linked structures, such as phenylalanyl 3′ deoxy 3′ amino adenosine, alanyl 3′ deoxy 3′ amino adenosine, and tyrosyl 3′ deoxy 3′ amino adenosine; in any of these compounds, any of the naturally-occurring L-amino acids or their analogs may be utilized. In addition, a combined tRNA-like 3′ structure-puromycin conjugate may also be used in the invention.

### Linker Molecules

In certain embodiments, the present invention relates to use a linker molecule which comprises: (1) a nucleic acid portion which hybridizes to the 3′ end of the mRNA produced by transcription of the DNA; (2) a peptide acceptor; and (3) a psoralen moiety. Optionally, a linker molecule may further comprise a non-nucleotide moiety.

(1) Nucleic Acid Portions

The term “nucleic acid portion” includes deoxyribonucleotides, ribonucleotides, analogs thereof, or a modified nucleic acid portion.

In one aspect of the invention a nucleic acid (e.g., a native mRNA or modified mRNA) may be attached to its encoded protein at the end of translation by the use of a nucleic acid or modified nucleic acid linker which is hybridized at the 3′ end of the nucleic acid. Accordingly, in some embodiments, the mRNA-protein fusion is formed by the interaction of the protein and the nucleic acid with the linker molecule.

In further embodiments, the nucleic acid portion of the linker molecule is capable of serving as a primer to reverse transcribe the mRNA.

### Non-Nucleotide Moieties

In some embodiments, the present invention employs one or more non-nucleotide moieties (separate from the nucleic acid portion as described above). In some embodiments, non-nucleotide moieties may be employed to connect a nucleic acid to a peptide acceptor. In other embodiments, non-nucleotide moieties may be used to connect a high affinity ligand (e.g., biotin) or a ligand acceptor (e.g., streptavidin) to a peptide acceptor. In another embodiment, non-nucleotide moieties may be used to connect a nucleic acid to a high affinity ligand.

In some specific embodiments, the non-nucleotide moieties are poly(alkylene oxide) moieties, which are a genus of compounds having a polyether backbone. Poly(alkylene oxide) species of use in the present invention may include, for example, straight- and branched-chain species. For example, poly(ethylene glycol) is a poly(alkylene oxide) consisting of repeating ethylene oxide subunits, which may or may not include additional reactive, activatable or inert moieties at either terminus. Derivatives of straight-chain poly(alkylene oxide) species that are heterobifunctional are also known in the art. In some embodiments, the non-nucleotide moieties may be composed of 5 to 50 subunits of poly(alkylene oxide), 10 to 30 subunits of poly(alkylene oxide), 10 to 20 units of poly(alkylene oxide), 15 to 20 units of poly(alkylene oxide), or, in some embodiments, 18 subunits of poly(alkylene oxide).

A poly(ethylene glycol) linker is a non-nucleotide moiety having a poly(ethylene glycol) (“PEG”) backbone or methoxy-PEG (“mPEG”) backbone, including PEG and mPEG derivatives. A wide variety of PEG and mPEG derivatives are known in the art and are commercially available. For example, Nektar, Inc. Huntsville, Ala., provides PEG and mPEG compounds useful as linkers or modifying groups optionally having nucleophilic reactive groups, carboxyl reactive groups, eletrophilically activated groups (e.g., active esters, nitrophenyl carbonates, isocyanates, etc.), sulfhydryl selective groups (e.g., maleimide), and heterofunctional (having two reactive groups at both ends of the PEG or mPEG), biotin groups, vinyl reactive groups, silane groups, phospholipid groups, and the like.

In Vitro Transcription Translation (IVTT) Expression Systems

The phrase “in vitro transcription translation” (IVTT), as used herein, refers to linked or coupled in vitro transcription/translation. In “conventional” in vitro translation systems, RNA is used directly as a template for translation. The RNA can be total RNA, mRNA, or a synthesized template. By contrast, “linked” or “coupled” in vitro transcription/translation systems start off with a DNA template. RNA is transcribed from the DNA template, subsequently translated into a protein without purification. Appropriate synthetic DNA templates for in vitro transcription/translation systems are readily generated, either after cloning into plasmid vectors or via PCR. Coding sequences are generally preceded by one of several bacteriophage promoters (T7, T3, or SP6) and translation control elements specific for the chosen prokaryotic or eukaryotic cell-free lysate system.

In “coupled” IVTT systems, transcription and translation occur in the same tube. Kits utilizing both prokaryotic and eukaryotic lysate systems are commercially available. In prokaryotic cells such as E. coli, transcription and translation occur simultaneously. During transcription, the 5′ end of RNA is available for ribosomal binding. Translation commences while the 3′ end is still being transcribed. This early RNA/ribosome interaction stabilizes transcripts and promotes efficient translation. Bacterial-based coupled IVTT systems are performed the same way, coupled in the same tube under the same reaction conditions. Bacterial-based coupled IVTT systems thus give efficient expression of either prokaryotic or eukaryotic gene products in a short amount of time. In eukaryotes, by contrast, transcription and translation occur sequentially in separate compartments at different times. Conditions that allow both transcription and eukaryotic translation to occur in a single tube may be suboptimal for one or both reactions. However, eukaryotic coupled IVTT systems have been successfully used and are commercially available. Examples of coupled IVTT systems include, but are not limited to, PURExpress™ In Vitro Protein Synthesis Kit (New England Biolab), TNT® Quick Coupled Transcription/Translation Systems (Promega), Rapid Translation System E. coli HY Kit (Roche), and PROTEINscript-PRO™ system (Ambion).

“Linked” IVTT systems involve two-step procedures. First, transcription is catalyzed with an appropriate polymerase. Second, a cell-free lysate system, usually eukaryotic, is used to effect translation. Because the transcription and translation reactions are separate in linked systems, each can be optimized. Examples of linked IVTT systems include, but are not limited to, EasyXpress™ Insect Protein Synthesis System (Qiagen) and Single Tube Protein™ System 3 (STP3) (Novagen).

The IVTT expression systems may comprise crude extracts from E. coli, wheat germ, or rabbit reticulocytes along with supplements required for efficient translation. The cell-free extracts contain ribosomes, aminoacyl-tRNA synthetases, along with all of the other requisite macromolecular components and factors for the translation of RNA. These extracts are generally supplemented with amino acids, energy sources, energy regeneration systems, and various cofactors. For example, the IVTT systems include the reagents required for transcription and translation, such as, primers, dNTPs, NTPs, tRNAs, amino acids, and appropriate enzymes. Specific components of the IVTT expression systems have been described in the art (e.g., Y. Shimizu et al., Methods 36 (2005) 299-304). Exemplary specific components of the IVTT expression systems are shown in Table 1.

TABLE 1
An example of the specific components
of the IVTT expression systems

Translation factors
2.7M IF1
0.40M IF2
1.5M IF3
0.26M EF-G
0.92M EF-Tu
0.66M EF-Ts
0.25M RF1
0.24M RF2
0.17M RF3
0.50M RRF
Energy sources
2 mM ATP, GTP
1 mM CTP, UTP
20 mM creatine phosphate
BuVers
50 mM Hepes-KOH, pH 7.6
100 mM potassium glutamate
13 mM magnesium acetate
2 mM spermidine
1 mM DTT
Aminoacyl-tRNA synthetases
1900 U/ml AlaRS
2500 U/ml ArgRS
20 mg/ml AsnRS
2500 U/ml AspRS
630 U/ml CysRS
1300 U/ml GlnRS
1900 U/ml GluRS
5000 U/ml GlyRS
630 U/ml HisRS
2500 U/ml IleRS
3800 U/ml LeuRS
3800 U/ml LysRS
6300 U/ml MetRS
1300 U/ml PheRS
1300 U/ml ProRS
1900 U/ml SerRS
1300 U/ml ThrRS
630 U/ml TrpRS
630 U/ml TyrRS
3100 U/ml ValRS
Other enzymes
4500 U/ml MTF
1.2M ribosomes
4.0 g/ml creatine kinase
3.0 g/ml myokinase
1.1 g/ml nucleoside-diphosphate kinase
2.0 units/ml pyrophosphatase
10 g/ml T7 RNA polymerase
Other components
0.3 mM 20 amino acids
10 mg/ml 10-formyl-5,6,7,8-tetrahydrofolic acid
56 A260/ml tRNAmix (Roche)

In certain embodiments of the invention, the present methods utilize an IVTT system wherein one or more translation components (e.g., tRNAs, amino acids, or ribosomes) are omitted from the original system, but are later added back into the system.

Examples of such IVTT systems include, but are not limited to, PURExpress® Δ (aa, tRNA) Kit (New England Biolab) and PURExpress® Δ Ribosome Kit (New England Biolab). Such IVTT systems allow greater control of the IVTT reaction and the photocross-linking reaction.

As one of skill in the art understands, suitable DNA molecules can be chosen or constructed as appropriate for a particular IVTT system. For example, the DNA molecules should contain appropriate regulatory sequences, including promoter sequences, terminator sequences, polyadenylation sequences, enhancer sequences, marker genes and other sequences as appropriate. Many known techniques and protocols for manipulation of nucleic acid, for example, in preparation of nucleic acid constructs, mutagenesis, sequencing, introduction of DNA into cells and gene expression, and analysis of proteins, are described in detail in Current Protocols in Molecular Biology, Second Edition, Ausubel et al. eds., John Wiley & Sons, 1992. The disclosures of Sambrook et al. and Ausubel et al. are incorporated herein by reference.

### Selection Methods

In some embodiments, protein or gene libraries may be screened to select proteins having desired qualities (e.g., binding to a particular antigen) or which have been improved or modified according to the methods of the invention. In related embodiments, a particular protein selected by the present methods may be further altered by affinity maturation or mutagenesis, thereby producing a library of related proteins or nucleic acids. As such, one aspect of the invention may involve screening large libraries in order to select potential proteins (or nucleic acids encoding said proteins) which have desirable qualities, such as the ability to bind an antigen, a higher binding affinity, etc. Any methods for library generation and target selection known in the art or described herein may be used in accordance with the present invention.

Methods of library generation known in the art, in accordance with the methods described herein (e.g., for adding the appropriate tags, complementary sequences, etc.) may be employed to create libraries suitable for use with the methods described herein. Some methods for library generation are described in U.S. Ser. No. 09/007,005 and 09/247,190; Szostak et al., WO989/31700; Roberts & Szostak (1997) 94:12297-12302; U.S. Ser. No. 60/110,549, U.S. Ser. No. 09/459,190, and WO 00/32823, which are incorporated herein by reference. In certain specific embodiments, the library is a VH or VL library.

In certain specific embodiments, the nucleic acid constructs of the library contain the T7 promoter. The nucleic acids in the library may be manipulated by any means known in the art to add appropriate promoters, enhancers, spacers, or tags which are useful for the production, selection, or purification of the nucleic acid, protein, or the fusion product. For example, in some embodiments, the sequences in the library may include a TMV enhancer, sequences encoding a FLAG tag, an SA display sequence, or a polyadenylation sequence or signal. In some embodiments, the nucleic acid library sequences may further include a unique source tag to identify the source of the RNA or DNA sequence.

The double stranded DNA library is then transcribed and translated in the IVTT system as described above in the presence of a linker molecule. As described above, the linker molecule (also referred to as the “puromycin linker”) comprises (i) a nucleic acid portion which hybridizes to the 3′ end of the mRNA encoded by the DNA molecule; (ii) a peptide acceptor; and (iii) a psoralen moiety. In some embodiments, the linker is photocrosslinked to the mRNA. In particular embodiments, a ligand acceptor, e.g., streptavidin, is then loaded. In further embodiments, a second high affinity ligand which is attached to a peptide acceptor is bound to the streptavidin. In some embodiments, the second high affinity ligand/peptide acceptor is a biotin-puromycin linker.

The mRNA-protein fusions may then undergo reverse transcription, optionally, without being purified. Reverse transcription generates a cDNA/RNA complex which may be noncovalently or covalently linked to the protein through association with the linker molecule.

Optionally, the resulting cDNA-RNA-protein fusion may then be treated with RNAse to degrade the remaining mRNA, followed by second strand DNA synthesis to generate a DNA-protein fusion. In a specific embodiment, the nucleic acid portion in the linker molecule may serve as a primer for reverse transcription.

After reverse transcription, the DNA-mRNA-protein fusions (also referred to as mRNA-protein fusions) may be further purified based on a tag which is engineered into the fusion. Any tag known in the art may be used, for example, a FLAG tag, a MYC tag, a Histidine tag (HIS tag), or a HA tag. In certain embodiments, a sequence encoding a FLAG tag is engineered into the original DNA sequence such that the encoded protein contains the FLAG tag. In certain specific embodiments, two different tag sequences (e.g., a FLAG tag and a HIS tag) are engineered into the original DNA sequence such that the encoded protein contains both tags.

The resulting mRNA-protein fusion is then selected for by using any selection method known in the art. In certain embodiments, affinity selection is used. For example, the desired binding target or antigen may be immobilized on a solid support for use in an affinity column. Examples of methods useful in affinity chromatography are described in U.S. Pat. Nos. 4,431,546, 4,431,544, 4,385,991, 4,213,860, 4,175,182, 3,983,001, 5,043,062, which are all incorporated herein by reference in their entirety. Subsequently, binding activity can be evaluated by standard immunoassay and/or affinity chromatography. Determining the ability of candidate proteins (e.g., antibodies, single chain antibodies, etc.) to bind therapeutic targets can be assayed in vitro using, e.g., a Biacore instrument, which measures binding rates of a protein to a given target.

In certain embodiments, the selected mRNA-protein fusions may be identified by sequencing of the nucleic acid component. Any sequencing technology known in the art may be employed, e.g., 454 Sequencing, Sanger sequencing, sequencing by synthesis, or the methods described in U.S. Pat. Nos. 5,547,835, 5,171,534, 5,622,824, 5,674,743, 4,811,218, 5,846,727, 5,075,216, 5,405,746, 5,858,671, 5,374,527, 5,409,811, 5,707,804, 5,821,058, 6,087,095, 5,876,934, 6,258,533, 5,149,625, which are all incorporated herein by reference in their entirety.

In some embodiments, the present selection methods may be performed multiple times to identify higher affinity binders, and may further be implemented with competitive binders or more stringent washing conditions. One of skill in the art will appreciate that variants of the procedure described herein may be employed.

### Uses of ASCENT Technology

The methods of the present invention have commercial applications in any area where protein technology is used to solve therapeutic or diagnostic problems. For example, the ASCENT technology is useful for improving or altering existing proteins as well as for isolating new proteins with desired functions. The proteins may be naturally-occurring sequences, altered forms of naturally-occurring sequences, or partly or fully synthetic sequences.

In certain embodiments, methods of the invention can be used to develop or improve polypeptides such as immunobinders, for example, antibodies, binding fragments or analogs thereof, single chain antibodies, catalytic antibodies, VL and/or VH regions, Fab fragments, Fv fragments, Fab′ fragments, Dabs, and the like. In certain specific embodiments, the methods will target the improvement of immunobinders, for example, regions of the variable region and/or CDRs of an antibody molecule, i.e., the structure responsible for antigen binding activity which is made up of variable regions of two chains, one from the heavy chain (VH) and one from the light chain (VL). Once the desired antigen-binding characteristics are identified, the variable region(s) can be engineered into an appropriate antibody class such as IgG, IgM, IgA, IgD, or IgE. It is understood that the methods may be employed to improve and/or select human immunobinders and/or immunobinders from other species, e.g., any mammalian or non-mammalian immunobinders, camelid antibodies, shark antibodies, etc.

In certain embodiments, methods of the invention may be used to improve or select human or humanized antibodies (or fragments thereof) for the treatment of diseases. For example, antibody libraries are developed and are screened in vitro, eliminating the need for techniques such as cell-fusion or phage display. The invention is also useful for improving single chain antibody libraries (Ward et al., Nature 341:544 (1989); and Goulot et al., J. Mol. Biol. 213:617 (1990)). For example, the variable region may be constructed either from a human source (to minimize possible adverse immune reactions of the recipient) or may contain a totally randomized cassette (to maximize the complexity of the library). To screen for improved antibody molecules, a pool of candidate molecules are tested for binding to a target molecule. Higher levels of stringency are then applied to the binding step as the selection progresses from one round to the next. Single chain antibodies may be used either directly for therapy or indirectly for the design of standard antibodies. Such antibodies have a number of potential applications, including the isolation of anti-autoimmune antibodies, immune suppression, and in the development of vaccines for viral diseases such as AIDS.

In certain embodiments, methods of the invention may be used to improve or select antibody derivatives or mimetics (e.g., Nanobodies, UniBodies, Adnectins, Affibodies, DARPins, Anticalins, Avimers, and Versabodies). Some of these antibody derivatives or mimetics are reviewed in Gill and Damle (2006) 17: 653-658, which is incorporated herein by reference. All of the antibody derivatives or mimetics may be improved and/or selected by the methods of the present invention. In some embodiments, methods known in the art to generate Nanobodies, UniBodies, Adnectins, Affibodies, DARPins, Anticalins, Avimers, and Versabodies may be used to discover an initial binding protein which may then serve as the basis for the generation of a library which may be produced and selected from according to the methods of the present invention. Alternatively, binders already known in the art may be used directly to create new libraries for use with the methods described herein.

In certain specific embodiments, methods of the invention will be used to improve or select adnectin molecules. Adnectin molecules are engineered binding proteins derived from one or more domains of the fibronectin protein (see, e.g., Lipovgek, 2011, Protein Eng Des Sel., 24(1-2): 3-9.). Typically, fibronectin is made of three different protein modules, type I, type II, and type III modules. For a review of the structure of function of the fibronectin, see Pankov and Yamada (2002) J Cell Sci.; 115(Pt 20):3861-3, Hohenester and Engel (2002) 21:115-128, and Lucena et al. (2007) Invest Clin. 48:249-262, which are incorporated herein by reference.

Depending on the originating tissue, fibronectin may contain multiple type III domains which may be denoted, e.g., 1FN3, 2FN3, 3FN3, etc. The 10FN3 domain contains an integrin-binding motif and further contains three loops which connect the beta strands. These loops may be thought of as corresponding to the antigen binding loops of the IgG heavy chain, and they may be altered by the present methods to select adnectin molecules that specifically bind a target of interest. Adnectin molecules to be improved may also be derived from polymers of 10FN3 related molecules rather than a simple monomeric 10FN3 structure.

10FN3 proteins adapted to become adnectin molecules are altered so to bind a target of interest. 10FN3 variant and mutant sequences (thereby forming a library) can be created by any method known in the art including, but not limited to, error prone PCR, site-directed mutagenesis, DNA shuffling, or other types of recombinational mutagenesis. In one example, variants of the DNA encoding a 10FN3 sequence may be directly synthesized in vitro. Alternatively, a natural 10FN3 sequence may be isolated or cloned from the genome using standard methods (as performed, e.g., in U.S. Pat. Application No. 20070082365, incorporated herein by reference), and then mutated using mutagenesis methods known in the art.

In one embodiment, a target protein may be immobilized on a solid support, such as a column resin or a well in a microtiter plate. The target is then contacted with a library of potential binding proteins as described herein. The library may comprise 10FN3 clones or adnectin molecules derived from the wild type 10FN3 by mutagenesis/randomization of the 10FN3 sequence or by mutagenesis/randomization of the 10FN3 loop regions. The selection/mutagenesis process may be repeated until binders with sufficient affinity to the target are obtained. Methods of generating libraries of altered 10FN3 domains and selecting appropriate binders may be carried out as described in the following U.S. patent and patent application documents and are incorporated herein by reference: U.S. Pat. Nos. 7,115,396; 6,818,418; 6,537,749; 6,660,473; 7,195,880; 6,416,950; 6,214,553; 6,623,926; 6,312,927; 6,602,685; 6,518,018; 6,207,446; 6,258,558; 6,436,665; 6,281,344; 7,270,950; 6,951,725; 6,846,655; 7,078,197; 6,429,300; 7,125,669; 6,537,749; 6,660,473; and U.S. Pat. Application Nos. 20070082365; 20050255548; 20050038229; 20030143616; 20020182597; 20020177158; 20040086980; 20040253612; 20030022236; 20030013160; 20030027194; 20030013110; 20040259155; 20020182687; 20060270604; 20060246059; 20030100004; 20030143616; and 20020182597. Also see the methods of the following references which are incorporated herein by reference in their entirety: Lipovsek et al et al. (2007) Journal of Molecular Biology 368: 1024-1041; Sergeeva et al. (2006) Adv Drug Deliv Rev. 58:1622-1654; Petty et al. (2007) Trends Biotechnol. 25: 7-15; Rothe et al. (2006) Expert Opin Biol Ther. 6:177-187; and Hoogenboom (2005) Nat. Biotechnol. 23:1105-1116.

Similarly, the ASCENT technology can be used for improving or altering other protein scaffolds and selecting binders with desired functions. For example, the following patent application documents relate to other protein scaffolds, and are incorporated herein by reference: WO 2011/130328; WO 2009/058379; WO 2011/130324; WO 2009/133208; WO 98/56919; WO 2011/051333; WO 2011/137319; WO 2010/093627; WO 2010/051274; and WO 2012/016245.

The present invention is further illustrated by the following examples which should not be construed as further limiting. The contents of all figures and all references, patents and published patent applications cited throughout this application are expressly incorporated herein by reference.

### Example 1

### Accelerated Profusion™ Selections of Adnectins

The ASCENT protocol is designed to enhance the current PROfusion™ protocol by simplifying and removing certain steps in order to allow the user to perform one round of selection in the span of approximately 4 hours. The process is designed such that all reactions prior to selection (e.g., transcription, translation, cross-linking, and reverse transcription) can be carried out in the same container (“one pot”), for example, a 96-well PCR plate, without any purification step between any of these reactions.

1. Library Creation

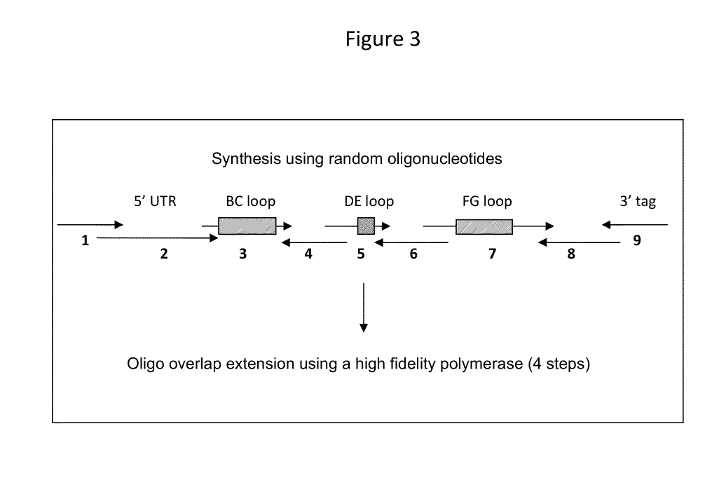

Certain libraries were constructed as described below and used in selections against targets of interest. As shown in FIG. 3, an Adnectin library was constructed by extension of overlapping oligonucleotides. Oligonucleotides were designed corresponding to the BC, DE and FG loops of 10NF3 flanked by fixed regions of the 10FN3 scaffold. Within each of the three loops, a certain amount of variability was designed using a specific distribution of trimer phosphoramidites.

The resulting Adnectin library contained the elements which are compatible with the ASCENT process, including the promoter for T7 RNA polymerase, and a 5′ untranslated region (5′UTR) which contains a spacer region and the Shine-Dalgarno ribosome binding site. The spacer region at the 5′UTR was composed primarily of a polyA sequence in order to compare the ASCENT process with the standard mRNA display process known as PROfusion™. An exemplary spacer region sequence is 5′-TAATACGACTCACTATAGGAAAAAAAAAAAAAAAAAAAAAGGAGGTATAC ATG (SEQ ID NO: 1). In addition, the 3′ end of the Adnectin library contained a C-terminal FLAG tag (DYKDDDDK; SEQ ID NO: 2) or a HIS6 tag (HHHHHH; SEQ ID NO: 3), or both tags in either order, followed by a short nucleotide sequence region which hybridizes to a puromycin-containing linker.

A library of over 10¹³ unique molecules was generated and selection was applied using ASCENT against a target of interest. During each selection round, purified binders were eluted in a buffer containing PBS and 0.025% Tween-20. After four to six rounds of selection, a sample of the amplified population was transformed into E. coli and subsequently grown, purified and assessed for the ability to bind to the target of interest.

2. In Vitro Transcription/Translation Reaction (about 2.25 Hours)

DNA libraries of good quality can be added to a cell-free mixture, which is reconstituted from the purified components necessary for E. coli transcription and translation components, to carry out in vitro transcription and translation (IVTT) in a one-step reaction. This reconstituted cell-free mixture is free of nucleases and proteases, which allows for preserving the integrity of DNA and RNA templates/complexes and resulting in proteins that are free of modification and degradation.

The one-step in vitro transcription/translation reaction can be carried out in two slightly different ways, which are shown in the following examples.

In the first example, the purified DNA and the puromycin linker are added into an IVTT reaction using the PURExpress kit (New England Biolabs, #E6800). The puromycin linker anneals to the 3′ end of the transcribed message, and subsequently incorporates into the translated peptide, creating a covalent bond between the linker and the peptide. Once the reaction has proceeded for 2 hours, the reaction can be removed and be incubated under long-wave length UV light (365 nm) for 15 minutes such that the linker can be covalently cross-linked to the mRNA. The reactions can be set up in a 96-well PCR plate and incubated in a PCR thermocycler.

In the second example, the purified DNA and the puromycin linker are added into an IVTT reaction using the delta PURExpress kit (-aa, -tRNA) (New England Biolabs, #E6840). During the first hour, the puromycin linker anneals to the 3′ end of the transcribed message. Once the reaction has proceeded for 1 hour, the linker can be covalently cross-linked to the mRNA by incubating under long wave length (365 nm) UV light for 15 minutes in a thin walled PCR tube. Then, the amino acids and tRNA mix, which are omitted from the delta kit, can be added to the reaction and incubate for an additional hour at 37° C. to generate mRNA/protein fusion. The reactions can be set up easily in a 96-well PCR plate and incubated in a PCR thermocycler.

3. Reverse Transcription Reaction (about 30 Minutes)

The IVTT reaction product does not need to be purified, and can be directly subject to the reverse transcription (RT) reaction. The RT reaction can be carried out, for example, using Superscript II (Invitrogen) at 42° C. for 30 minutes. This reaction reverse transcribes mRNA into DNA, creating the mRNA/DNA fusion protein (also known as a fusion molecule). This reaction can also be done in the same 96-well PCR plate.

4. HIS or FLAG Purification (about 30 Minutes)

After the RT reaction, the resulting fusion molecules can then be purified based on the HIS6 tag or the FLAG tag. For example, fusion molecules can be purified using Ni-NTA agarose from QIAGEN. Some of the reconstituted translation components are made with HIS tags and may be co-purified as well. An excess of Ni-NTA agarose is utilized to ensure sufficient recovery. Alternatively, fusion molecules can be purified using an anti-FLAG antibody (e.g., the M2 antibody from Sigma) coupled to agarose beads for 30 minutes at room temperature and eluted using FLAG peptide.

5. Selection

After HIS or FLAG purification, the resulting fusion molecules can be selected against a target of interest, and the surviving molecules can be amplified via the DNA tail.

6. Quantitative PCR (qPCR) Analysis

The ASCENT-qPCR-F primer
(5′-AAGGAGGTATACATG-3′; SEQ ID NO: 4)
and
the ASCENT-qPCR-R primer
(5′-GTCGTCGTCCTTGTAGTC-3′; SEQ ID NO: 5)

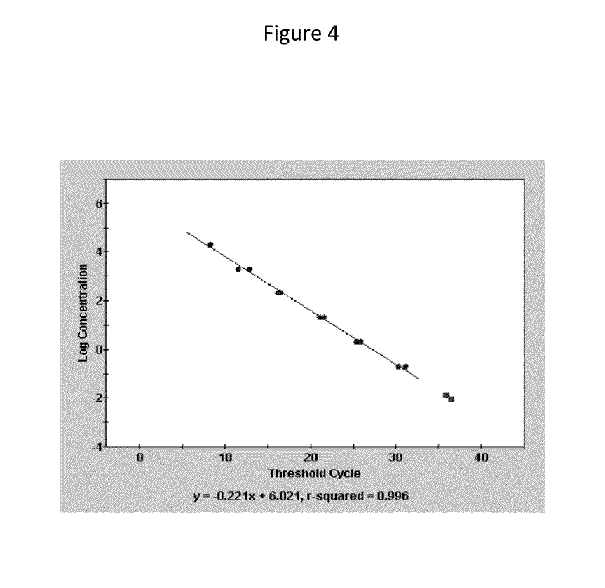

are examples primers which are compatible with an ASCENT library. Each of these primers was used at 0.5 uM final concentration in the qPCR analysis. The standard curve is shown in FIG. 4, with units in amol (y=−0.221x+6.021, r̂2=.996).

7. PCR Amplification (about 2 Hours)

After the selection step, the identified potential binders can be PCR-amplified via the DNA tail and subject to further rounds of the ASCENT process as described above.

An exemplary PCR reaction is described below. DNA from a particular post-round PCR is amplified using KOD polymerase with proper 5′ and 3′ ends to include E. coli 5′UTR, and the 3′ HIS6 tag and/or the 3′ FLAG tag. The following two primers (T7ATG-12 5′ primer and Flag_HIS6-R 3′ primer) are example primers that would work with libraries constructed with the above-described 5′UTR and 3′ HIS6 tag and/or the 3′ FLAG tag.

T7ATG-12 5′ primer
(SEQ ID NO: 6):
5′-TAATACGACTCACTATAGGAAAAAAAAAAAAAAAAAAAAAGGAGGT
ATACATG-3′
Flag_HIS6-R 3′primer
(SEQ ID NO: 7):
5′-TTAAATAGCGGATGCGTGGTGGTGGTGATGGTGCTTGTCGTCGTCG
TCCTTGTAGTC-3′

### Example 2

### Analysis of Fusion Yield

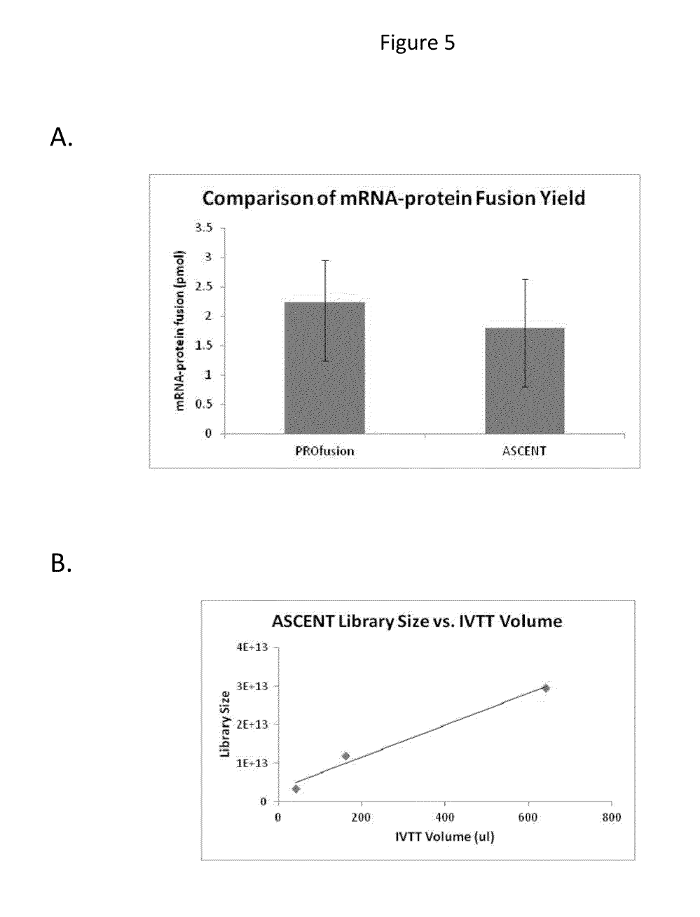

Libraries based upon the human fibronectin 10th type III domain were generated using a combination of degenerate oligonucleotides and trimer phosphoramidites to generate variation within the various binding loops (see Example 1). mRNA-protein fusions were generated from the DNA libraries using both ASCENT and PROfusion™ Because there is no ribosomal turnover during the in vitro translation step, the size of the mRNA-protein fusion library is typically limited by the number of ribosomes in the reaction. Therefore, larger libraries can be generated by scaling the volume of the IVTT reaction, which increases the number of ribosomes available for translation. Using a 40-ul scale IVTT reaction for both PROfusion™ and ASCENT, the amount of mRNA-protein fusion generated after the HIS or FLAG purification was measured using quantitative PCR (qPCR). An average mRNA-protein fusion yield was calculated. As shown in FIG. 5, PROfusion™ and ASCENT technologies demonstrated comparable yields (FIG. 5A), and the fusion yield scaled with the IVTT volume (FIG. 5B), suggesting that the ASCENT process could generate libraries on the order of 10¹² to 10¹³ if desired.

### Example 3

### Analysis of Enrichment

The ability of a selection technology to enrich a binding population from a diverse starting library is dependent upon the enrichment rate of that particular binding population during subsequent rounds of selection. Libraries of 10¹² to 10¹³ unique molecules will likely only contain a very small fraction of target-specific binders. While the exact numbers are likely dependent upon the particular composition of the starting library and the complexity of the selection target, in many cases the number of unique binders with sub-micromolar affinity is less than 1 million. In order to enrich this particular binding pool such that multiple copies exist in a sample of 100 sequenced clones after 4-6 rounds of selection, the enrichment rate should generally be greater than 50 fold each round. Very typically, binders with very tight affinities do not bind to the target at 100%. Therefore, enrichment rates less than 50 fold run the risk of not covering all of the unique molecules identified during the first round of selection.

In order to test the enrichment rate during ASCENT, a very simple library containing only two starting clones was generated. Both clones had been previously identified using PROfusion™. Clone 1 is a human serum albumin (HuSA) binder and clone 2 is an interleukin-23 (IL-23) binder. Four different mixtures were generated based on various ratios of clone 1 to clone 2. Library 1 contained a 1:1 mixture of clone 1 to clone 2. Library 2 contained a 1:100 mixture of clone 1 to clone 2. Library 3 contained a 1:10,000 mixture of clone 1 to clone 2. Library 4 contained a 1:1,000,000 mixture of clone 1 to clone 2. Selections were performed using ASCENT against IL-23, and the binding percentage was calculated each round.

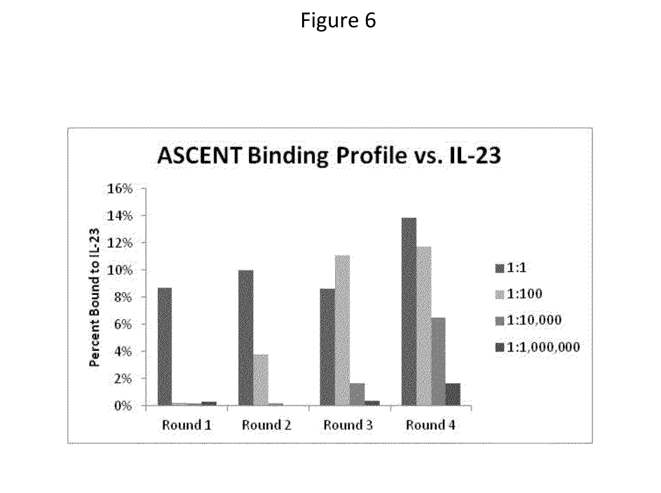

The results are shown in FIG. 6. In round 1 (R1), Library 1 yielded approximately 8% binding against IL-23, while Libraries 2-4 yielded binding percentages close to background levels. However, in the later rounds (R2-R4), positive binding signals were observed due to enrichment of clone 2, as determined by sequence data of the surviving binding pools. These results are consistent with an approximately 50-fold enrichment rate, suggesting that ASCENT could theoretically select for a population of binders from a diverse starting library in the typical 4-6 rounds of selection.

### EQUIVALENTS

Those skilled in the art will recognize or be able to ascertain, using no more than routine experimentation, many equivalents of the specific embodiments of the invention described herein. Such equivalents are intended to be encompassed by the following claims.

We claim:

1. A method for generating an mRNA-protein fusion molecule, comprising:
(a) contacting an in vitro transcription translation (IVTT) system with (1) a DNA molecule which comprises a protein coding sequence and a 5′ untranslated region (5′ UTR); and (2) a linker molecule which comprises (i) a nucleic acid portion which hybridizes to the 3′ end of the mRNA encoded by the DNA molecule; (ii) a peptide acceptor; and (iii) a psoralen moiety;
(b) transcribing the DNA molecule into an mRNA and translating the mRNA into a protein in the IVTT system, wherein the protein is fused to the mRNA through the linker molecule; and
(c) cross-linking the mRNA-protein fusion through the psoralen moiety.

2. The method of claim 1, further comprising reverse-transcribing the mRNA fused to the protein, thereby generating a DNA-mRNA-protein fusion molecule.

3. The method of claim 1, wherein the IVTT system is an Escherichia coli based IVTT system.

4. The method of claim 1, wherein all steps are performed in the same container.

5. The method of claim 1, wherein the linker molecule further comprises a non-nucleotide moiety.

6. The method of claim 1, wherein the peptide acceptor is puromycin.

7. The method of claim 1, wherein the psoralen moiety is positioned at the 5′ terminus or the 3′ terminus of the linker molecule.

8. The method of claim 1, wherein the cross-linking is achieved by UV light irradiation.

9. The method of claim 1, wherein the protein is an antibody or an antibody fragment.

10. The method of claim 2, wherein the mRNA-protein fusion is not purified before the reverse transcription step.

11. The method of claim 1, wherein the mRNA produced by transcription is not purified before translation.

12. The method of claim 1, wherein the DNA molecule further comprises at least a nucleic acid sequence which encodes a tag.

13. The method of claim 12, wherein the tag is selected from histidine and FLAG.

14. A method for selecting a desired protein, comprising:
(a) contacting an in vitro transcription translation (IVTT) system with:
(1) a population of DNA molecules, each of which comprises a protein coding sequence and a 5′ untranslated region (5′ UTR); and
(2) a linker molecule which comprises (i) a nucleic acid portion which hybridizes to the 3′ end of the mRNAs encoded by the DNA molecules; (ii) a peptide acceptor; and (iii) a psoralen moiety;
(b) transcribing the population of DNA molecules into a population of mRNAs and translating the population of the mRNAs into a population of proteins in the IVTT system, wherein each protein is fused to its encoding mRNA;
(c) cross-linking the mRNA-protein fusions through the psoralen moiety; and
(d) reverse-transcribing the mRNAs, thereby generating the DNA-mRNA-protein fusions; and
(e) selecting a desired mRNA-protein fusion, thereby selecting the desired protein.

15. The method of claim 14, wherein the DNA-mRNA-protein fusions are purified before the selection step.

16. A method for generating an mRNA-protein fusion molecule, comprising:
(a) contacting an in vitro transcription translation (IVTT) system which is deficient in one or more translation components with (1) a DNA molecule which comprises a protein coding sequence and a 5′ untranslated region (5′ UTR); and (2) a linker molecule which comprises (i) a nucleic acid portion which hybridizes to the 3′ end of the mRNA encoded by the DNA molecule; (ii) a peptide acceptor; and (iii) a psoralen moiety;
(b) transcribing the DNA molecule into an mRNA in the IVTT system, wherein the linker molecule anneals to the 3′ end of the mRNA;
(c) cross-linking the linker molecule and the mRNA through the psoralen moiety; and
(d) translating the mRNA into a protein by adding the one or more translation components into the IVTT system, wherein the protein is fused to the mRNA through the linker molecule.

17. The method of claim 16, further comprising reverse-transcribing the mRNA fused to the protein, thereby generating a DNA-mRNA-protein fusion molecule.

18. The method of claim 16, wherein the IVTT system is deficient in one or more translation components selected from amino acids, tRNAs, and ribosomes.

19. A method for selecting a desired protein, comprising:
(a) contacting an in vitro transcription translation (IVTT) system which is deficient in one or more translation components, with:
(1) a population of DNA molecules, each of which comprises a protein coding sequence and a 5′ untranslated region (5′ UTR); and
(2) a linker molecule which comprises (i) a nucleic acid portion which hybridizes to the 3′ end of each of the mRNAs encoded by the DNA molecule; (ii) a peptide acceptor; and (iii) a psoralen moiety;
(b) transcribing the population of DNA molecules into a population of mRNAs in the IVTT system, wherein the linker molecule anneals to the 3′ end of each of mRNAs;
(c) cross-linking the linker molecule and each of the mRNAs through the psoralen moiety; and
(d) translating the population of mRNAs into a population of proteins by adding the one or more translation components into the IVTT system, wherein each protein is fused to its encoding mRNA; and
(e) reverse-transcribing the mRNAs, thereby generating the DNA-mRNA-protein fusions; and
(f) selecting a desired mRNA-protein fusion, thereby selecting the desired protein.

20. The method of claim 19, wherein the IVTT system is deficient in one or more translation components selected from amino acids, tRNAs, and ribosomes.
